SEQUENCE LISTING (8 sequences)

SEQ ID NO: 1 - DNA, 24 bp, Artificial Sequence
  MUC4 rs774527434 SNP forward primer
ctttcttcagcttccacagatgac

SEQ ID NO: 2 - DNA, 18 bp, Artificial Sequence
  MUC4 rs774527434 SNP reverse primer
tggatgccgaggaaacgt

SEQ ID NO: 3 - DNA, 15 bp, Artificial Sequence
  MUC4 rs774527434 SNP reporter 1
accacccgtcttcct

SEQ ID NO: 4 - DNA, 15 bp, Artificial Sequence
  MUC4 rs774527434 SNP reporter 2
accacccatcttcct

SEQ ID NO: 5 - DNA, 21 bp, Artificial Sequence
  MUC4 rs531395109 SNP forward primer
gccatcgcatctgaagtaagc

SEQ ID NO: 6 - DNA, 25 bp, Artificial Sequence
  MUC4 rs531395109 SNP reverse primer
ggttgctttctgtgttaatctgtgt

SEQ ID NO: 7 - DNA, 15 bp, Artificial Sequence
  MUC4 rs531395109 SNP reporter 1
ttcagcgtgctcacg

SEQ ID NO: 8 - DNA, 16 bp, Artificial Sequence
  MUC4 rs531395109 SNP reporter 2
cttcagcatgctcacg

(19) United States
(12) Patent Application Publication
(10) Pub. No.: US 2023/0348989 A1
(43) Pub. Date: Nov. 2, 2023

(54) COMPOSITION COMPRISING MUC4 GENE MUTATION DETECTING AGENT FOR PREDICTION OR DIAGNOSIS OF GASTRIC CANCER

(71) Applicant: Seoul National University Hospital, Seoul (KR)

(72) Inventor: Nayoung Kim, Seongnam-Si (KR)

(21) Appl. No.: 18/015,198

(22) PCT Filed: Jul. 9, 2021
(86) PCT No.: PCT/KR2021/008803

§ 371 Date: Jul. 3, 2023

(30) Foreign Application Priority Data
Jul. 10, 2020 (KR) 10-2020-0085614
Aug. 13, 2020 (KR) 10-2020-0101967

(51) Int. Cl. C12Q 1/6886

(52) U.S. Cl. CPC C12Q 1/6886; C12Q 2600/156; C12Q 2600/158

(57) ABSTRACT

The present invention in which a MUC4 gene is discovered as a biomarker for predicting or diagnosis gastric cancer relates to a composition and kit capable of predicting or diagnosing gastric cancer when a mutation is present on the gene and to a method for providing information therefor. The composition according to an aspect of the present invention allows the detection of a mutation at one or more loci selected from the group consisting of rs774527434, rs534579185, rs77250903, rs868067409, rs531395109, rs754808151, rs1304612772, rs774907241, rs771925912, rs745342765, rs148735556, rs11717039, and rs547775645 on MUC4 gene, thus exhibiting an excellent effect of predicting or diagnosing gastric cancer in a cost and time effective manner for multiple subjects to be tested.

### TECHNICAL FIELD

Disclosed herein is a composition, kit, and information providing method capable of discovering MUC4 gene as a biomarker for predicting or diagnosing gastric cancer, and predicting or diagnosing gastric cancer when there is a mutation in the MUC4 gene.

### BACKGROUND ART

Gastric cancer (GC) is one of the most common cancers and the third leading cause of cancer mortality worldwide, with an estimated 783,000 deaths in 2018 [Bray F, Ferlay J. Soerjomataram I, Siegel RL, Torre LA, Jemal A. Global cancer statistics 2018: GLOBOCAN estimates of incidence and mortality worldwide for 36 cancers in 185 countries. CA Cancer J Clin. 2018; 68(6):394-424. Epub 2018/09/13. https://doiorg/10.3322/caac.21492 PMID: 30207593]. Korea has the highest incidence of gastric cancer in the world. In addition to males, Helicobacter pylori (H. pylori) infection, smoking, and frequent salty and nitrite diets, family history is a well-known risk factor for gastric cancer. [Parsonnet J, Friedman GD, Orentreich N, Vogelman H. Risk for gastric cancer in people with CagA positive or CagA negative Helicobacter pylori infection. Gut. 1997; 40(3):297-301. Epub 1997/03/01. https://doi.org/10.1136/gut.40.3.297 PMID: 9135515; PubMed Central PMCID: PMC1027076]. Most gastric cancers occurs sporadically, and only about 90% of cases in the community have an average risk [La Vecchia C, Negri E, Franceschi S, Gentile A. Family history and the risk of stomach and colorectal cancer. Cancer. 1992; 70(1):50-5. Epub 1992/07/01. https://doi.org/10.1002/1097-0142(19920701) 70:1<50::aid-cncr2820700109>3.0.co;2-i PMID: 1606546][Zanghieri G, Di Gregorio C, Sacchetti C, Fante R, Sassatelli R, Cannizzo G, et al. Familial occurrence of gastric cancer in the 2-year experience of a population-based registry. Cancer. 1990; 66(9):2047-51. Epub 1990/11/01. https://doi.org/10.1002/1097-0142(19901101)66:9<2047::aid-cncr2820660934>3.0. co;2-g PMID: 2224804]. Hereditary cancer syndromes, including hereditary diffuse gastric cancer (HDGC), account for less than 3% of all gastric cancer cases. The remaining 7% of cases are found in individuals with undiagnosed family history of hereditary cancer syndrome [McLean MH, El-Omar EM. Genetics of gastric cancer. Nat Rev Gastroenterol Hepatol. 2014; 11 (11):664-74. Epub 2014/08/20. https://doi.org/10.1038/nrgastro.2014.143 PMID: 25134511].

Individuals with first-degree relatives (FDRs) with gastric cancer have a 2 to 3-fold increased risk of gastric cancer [Choi YJ, Kim N. Gastric cancer and family history. Korean J Intern Med. 2016; 31(6):1042-53. Epub 2016/11/04. https://doi.org/10.3904/kjim.2016.147 PMID: 27809451; PubMed Central PMCID: PMC5094936]. The increased risk in families with gastric cancer is due in part to sharing of similar environmental factors, such as dietary habits or Helicobacter pylori infection. Nevertheless, the frequently observed weak association between Helicobacter pylori infection and gastric cancer incidence in families with gastric cancer suggests a genetic basis for familial aggregation. [Choi YJ, Kim N, Jang W, Seo B, Oh S, Shin CM, et al. Familial Clustering of Gastric Cancer: A Retrospective Study Based on the Number of First-Degree Relatives. Medicine (Baltimore). 2016; 95(20): e3606. Epub 2016/05/20. https://doi.org/10.1097/MD.0000000000003606 PMID: 27196462; PubMed Central PMCID: PMC4902404][Sahasrabudhe R, Lott P, Bohorquez M, Toal T, Estrada AP, Suarez JJ, et al. Germline Mutations in PALB2, BRCA1, and RAD51C, Which Regulate DNA Recombination Repair, in Patients With Gastric Cancer. Gastroenterology. 2017; 152(5):983-6 e6. Epub 2016/12/28. https://doi.org/10.1053/j.gastro. 2016.12.010 PMID: 28024868; PubMed Central PMCID: PMC5367981].

According to the literatures, several SNPs associated with gastric cancer have been identified through candidate gene approaches [ean MH, El-Omar EM. Genetics of gastric cancer. Nat Rev Gastroenterol Hepatol. 2014; 11 (11):664-74. Epub 2014/08/20. https://doi.org/10.1038/nrgastro.2014.143 PMID: 25134511][El-Omar EM, Carrington M, Chow WH, McColl KE, Bream JH, Young HA, et al. Interleukin-1 polymorphisms associated with increased risk of gastric cancer. Nature. 2000; 404(6776):398-402. Epub 2000/04/04. https://doi.org/10.1038/35006081 PMID: 10746728], or genome-wide association studies (GWAS) [Saeki N, Saito A. Choi U, Matsuo K, Ohnami S, Totsuka H, et al. A functional single nucleotide polymorphism in mucin 1, at chromosome 1q22, determines susceptibility to diffuse-type gastric cancer. Gastroenterology. 2011; 140(3):892-902. Epub 2010/11/13. https://doi.org/10.1053/j.gastro.2010.10.058 PMID: 21070779][Study Group of Millennium Genome Project for C, Sakamoto H, Yoshimura K, Saeki N, Katai H, Shimoda T, et al. Genetic variation in PSCA is associated with susceptibility to diffuse-type gastric cancer. Nat Genet. 2008; 40(6):730-40. Epub 2008/05/20. https://doi.org/10.1038/ng.152 PMID: 18488030]. One of the best known is the association of MUC1 with gastric cancer [Saeki N, Sakamoto H, Yoshida T. Mucin 1 gene (MUC1) and gastric-cancer susceptibility. Int J Mol Sci. 2014; 15(5):7958-73. Epub 2014/05/09. https://doi.org/10.3390/ijms15057958 PMID: 24810688; PubMed Central PMCID: PMC4057712]. MUC1 belongs to the mucin family and is located on the apical surface of mucosal epithelial cells and acts as a protective barrier against extrinsic damage. It is hypothesized that MUC1 mutations such as rs4072037 affect the quantity and quality of MUC1 protein, and that differences in gastric cancer susceptibility among individuals cause differences in barrier function in the stomach. [Saeki N, Sakamoto H, Yoshida T. Mucin 1 gene (MUC1) and gastric-cancer susceptibility. Int J Mol Sci. 2014; 15(5):7958-73. Epub 2014/05/09. https://doi.org/10.3390/ijms15057958 PMID: 24810688;PubMed Central PMCID: PMC4057712].

However, studies of these SNPs have yielded inconsistent results, especially with regard to different gastric cancer types and ethnicities [Saeki N, Saito A, Choi IJ, Matsuo K, Ohnami S, Totsuka H, et al. A functional single nucleotide polymorphism in mucin 1, at chromosome 1q22, determines susceptibility to diffuse-type gastric cancer. Gastroenterology. 2011; 140(3):892-902. Epub 2010/11/13. https://doi.org/10.1053/j.gastro.2010.10.058 PMID: 21070779][El-Omar EM, Rabkin CS, Gammon MD, Vaughan TL, Risch HA, Schoenberg JB, et al. Increased risk of noncardia gastric cancer associated with proinflammatory cytokine gene polymorphisms. Gastroenterology. 2003; 124(5):1193-201. Epub 2003/05/06. https://doi.org/10.1016/s0016-5085(03)00157-4 PMID: 12730860][Kim N, Cho SI, Yim JY, Kim JM, Lee DH, Park JH, et al. The effects of genetic polymorphisms of IL-1 and TNF-A on Helicobacter pylori-induced gastroduodenal diseases in Korea. Helicobacter. 2006; 11 (2):105-12. Epub 2006/04/04. https://doi.org/10.1111/j.1523-5378.2006.00384.x PMID: 16579840][Palmer AJ, Lochhead P, Hold GL, Rabkin CS, Chow WH, Lissowska J, et al. Genetic variation in C20orf54, PLCE1 and MUC1 and the risk of upper gastrointestinal cancers in Caucasian populations. Eur J Cancer Prev. 2012; 21(6):541-4. Epub 2012/07/19. https://doi.org/10.1097/CEJ. 0b013e3283529b79 PMID: 22805490; PubMed Central PMCID: PMC3460062]. Moreover, the effect sizes of SNPs obtained from GWAS were generally small, less than 2.0. Recently, novel gastric cancer genes, including PALB2, BRCA1, and CTNNA1, which account for a small fraction of familial gastric cancer, were identified using whole-genome and whole-exome sequencing (WES). [Sahasrabudhe R, Lott P, Bohorquez M, Toal T, Estrada AP, Suarez JJ, et al. Germline Mutations in PALB2, BRCA1, and RAD51C, Which Regulate DNA Recombination Repair, in Patients With Gastric Cancer. Gastroenterology. 2017; 152(5):983-6 e6. Epub 2016/12/28. https://doiorg/10.1053/j.gastro. 2016.12.010 PMID: 28024868; PubMed Central PMCID: PMC5367981][Fewings E, Larionov A, Redman J, Goldgraben MA, Scarth J, Richardson S, et al. Germline pathogenic variants in PALB2 and other cancer-predisposing genes in families with hereditary diffuse gastric cancer without CDH1 mutation: a whole-exome sequencing study. Lancet Gastroenterol Hepatol. 2018; 3 (7):489-98. Epub 2018/05/01. https://doi.org/10.1016/S2468-1253(18)30079-7 PMID: 29706558; PubMed Central PMCID: PMC5992580][Majewski IJ, Kluijt I, Cats A, Scerri TS, de Jong D, Kluin RJ, et al. An alpha-E-catenin (CTNNA1) mutation in hereditary diffuse gastric cancer. J Pathol. 2013; 229(4):621-9. Epub 2012/12/05. https://doi.org/10.1002/path.4152 PMID: 23208944]. Although most previous studies on familial clustering of gastric cancer have focused on HDGC or diffuse-type gastric cancer, [Sahasrabudhe R, Lott P, Bohorquez M, Tool T, Estrada AP, Suarez JJ, et al. Germline Mutations in PALB2, BRCA1, and RAD51C, Which Regulate DNA Recombination Repair, in Patients With Gastric Cancer. Gastroenterology. 2017; 152(5):983-6 e6. Epub 2016/12/28. https://doi.org/10.1053/j.gastro. 2016.12.010 PMID: 28024868; PubMed Central PMCID: PMC5367981][Fewings E, Larionov A, Redman J, Goldgraben MA, Scarth J, Richardson S, et al. Germline pathogenic variants in PALB2 and other cancer-predisposing genes in families with hereditary diffuse gastric cancer without CDH1 mutation: a whole-exome sequencing study. Lancet Gastroenterol Hepatol. 2018; 3 (7):489-98. Epub 2018/05/01. https://doi.org/10.1016/S2468-1253(18)30079-7 PMID: 29706558; PubMed Central PMCID: PMC5992580], a significant proportion of intestinal-type gastric cancer occurs in gastric cancer familial clusters. [Choi YJ, Kim N, Jang W, Seo B, Oh S, Shin CM, et al. Familial Clustering of Gastric Cancer: A Retrospective Study Based on the Number of First-Degree Relatives. Medicine (Baltimore). 2016; 95(20): e3606. Epub 2016/05/20. https://doi.org/10.1097/MD.0000000000003606 PMID: 27196462; PubMed Central PMCID: PMC4902404][Kaurah P, MacMillan A, Boyd N, Senz J, De Luca A, Chun N, et al. Founder and recurrent CDH1 mutations in families with hereditary diffuse gastric cancer. JAMA. 2007; 297(21):2360-72. Epub 2007/06/05. https://doi.org/10.1001/jama.297.21.2360 PMID: 17545690]. In addition, WES studies on gastric cancer are rare in Asia, where the prevalence of intestinal-type gastric cancer is high.

### DISCLOSURE

### Technical Problem

The present inventors recruited both family members with gastric cancer and family members without gastric cancer and identified MUC4 as a candidate predisposing gene with a large effect. The present inventors further performed the verification through the analysis of MUC4 expression in normal gastric mucosa and gastric cancer tissue in groups with a large number of cases and controls.

Accordingly, in one aspect, an object of the present invention is to provide a biomarker for predicting or diagnosing gastric cancer.

In another aspect, an object of the present invention is to provide a biomarker mutation for predicting or diagnosing gastric cancer.

### Technical Solution

In one aspect, the present invention provides a composition for predicting or diagnosing gastric cancer comprising a detecting agent of a mutation of a mucin 4 (MUC4) gene, wherein the mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on the MUC4 gene.

In another aspect, the present invention provides a kit for predicting or diagnosing gastric cancer, comprising the composition.

In another aspect, the present invention provides a method for providing information for predicting or diagnosing gastric cancer comprising extracting genomic DNA from a sample of a subject; and detecting a mutation at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on a mucin 4 (MUC4) gene in the extracted genomic DNA.

### ADVANTAGEOUS EFFECTS

In one aspect, the present invention has discovered the MUC4 gene as a biomarker for predicting or diagnosing gastric cancer, and identified that, in the case of gastric cancer patients unlike normal controls, mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 of the MUC4 gene, and in particular, in a subject with a MUC4 germline missense mutation (rs547775645 missense mutation), the expression is downregulated in noncancerous gastric mucosa, causing gastric cancer by loss of function of MUC4, and the expression of MUC4 is increased in cancer tissues compared to normal tissues. Therefore, the composition comprising the detecting agent of the mutation of the MUC4 gene has excellent effects of predicting or diagnosing gastric cancer cost-effectively and time-effectively for a large number of subjects.

### DESCRIPTION OF DRAWINGS

FIG. 1 shows a pedigree of a family with a MUC4 mutation according to one aspect of the present invention. In FIG. 1, subjects whose DNA was analyzed according to one aspect of the present invention are indicated by numbers beginning with “#”, and their ages at the time of diagnosis of gastric cancer were indicated in parentheses after GC. An arrow in FIG. 1 indicates a proband, a cross indicates a deceased subject, and a mut indicates a subject with a gene mutation. #34 of the subjects had c.5005A>G; another MUC4 mutation of p.S1669G. Abbreviations in FIG. 1 are as follows: GC, gastric cancer (not known by Lauren classification); IGC, intestinal-type gastric cancer; DGC, diffuse-type gastric cancer; RCC, renal cell cancer; ca, cancer; TA, tubule adenoma; F, family.

FIGS. 2A to 2C show predisposition gene candidates for gastric cancer obtained by combining pVAAST and linkage analysis according to one aspect of the present invention. FIG. 2A shows detected predisposing genes wherein a complex likelihood ratio test (CLRT) based on the binomial likelihood for the number of alleles in gastric cancer cases and controls was performed, which was weighted by the functional prediction likelihood ratio performed with 10⁶ substituted samples by the gene drop method. FIG. 2B shows a Manhattan plot of the LOD p values of all protein-encoding genes obtained from performing pVAAST. In FIG. 2B, each point on the plot represents the p value for one gene, and the x-axis shows the location of the genome arranged on the chromosome. FIG. 2C shows a quantile-quantile (QQ) plot of LOD p values obtained from pVAAST.

FIG. 3 shows a representative photomicrograph of IHC for MUC4 in noncancerous and cancerous gastric mucosal tissues of Family No. 14 (A-F: original magnification, H and I: X400). In FIG. 3, A is the tissue of #50 (subject number); B and C are tissues of #51; D is the tissue of #54; E and F are the tissues of #52; H and I are the tissues of #53. The staining intensity (brown) of MUC4 in non-cancerous tissue (A, D) of representative MUC4 mutation-negative controls is compared with absence or faint immunoreactivity of non-cancerous tissue in MUC4 mutation-positive gastric cancer patients (B, E and H) and shown. A pair of cancer tissues (C, F and I) in B, E and H shows strong and diffused staining of MUC4. MUC4 immune strength in MUC4 mutation-positive noncancerous mucosa was weak compared to MUC4 mutation-negative (left graph in G). Despite the increasing trend of immune strength in MUC4 mutation-positive cancer tissues, no statistical significance was observed (right graph in G). The white bar in G indicates MUC4 mutation-negative and a black bar indicates MUC4 mutation-positive.

FIG. 4 shows a QQ plot of LOD p values of genes MUC4, MAGEC1, and RETSAT according to an aspect of the present invention.

### MODE FOR INVENTION

Hereinafter, the present invention will be described in detail.

In one aspect of the present invention, the term “mutant” includes those in which the nucleotide and amino acid sequence of a corresponding gene include base substitution, deletion, insertion, amplification, and rearrangement. The nucleotide modification indicates a change in the nucleotide sequence with respect to a reference sequence (e.g., a wild-type sequence) (e.g., insertion, deletion, inversion, or substitution of one or more nucleotides, such as single-nucleotide polymorphism (SNP)). This term, unless otherwise indicated, may also include changes in the complement of the nucleotide sequence. The nucleotide modification may be a somatic mutation or germline polymorphism. In this specification, mutant may be used interchangeably with variant.

Additionally, in one aspect of the present invention, the amino acid modification may indicate a change in the amino acid sequence with respect to the reference sequence (e.g., a wild-type sequence) (e.g., insertion, substitution, or deletion of one or more amino acids, such as internal deletion or N- or C-terminus truncation).

Additionally, in one aspect of the present invention, the “gastric cancer prediction” may refer to predicting or diagnosing whether a patient has a risk for gastric cancer, whether the risk for gastric cancer is relatively high, what the cause of gastric cancer is, or whether gastric cancer has already occurred. Additionally, in one aspect of the present invention, the “gastric cancer diagnosis” may refer to confirming presence or features of pathological conditions. On the purpose of one aspect of the present invention, diagnosis may refer to confirming whether gastric cancer has occurred. The composition, kit or method according to one aspect of the present invention may be used to delay the onset or prevent the occurrence of gastric cancer through special and appropriate care for a specific patient, who is a patient having a high risk of developing gastric cancer. In addition, the composition, kit or method according to one aspect of the present invention may be clinically used to determine treatment by selecting the most appropriate treatment method through early diagnosis of gastric cancer.

In one aspect, the present invention provides a composition for predicting or diagnosing gastric cancer comprising a detecting agent of a mutation of a mucin 4 (MUC4) gene, wherein the mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on the MUC4 gene.

The MUC4 gene mutation according to one aspect of the present invention may suppress expression of the MUC4 gene in noncancerous gastric mucosa.

In addition, the MUC4 gene mutation according to one aspect of the present invention may increase the expression of the MUC4 gene in gastric cancer tissue compared to normal gastric tissue.

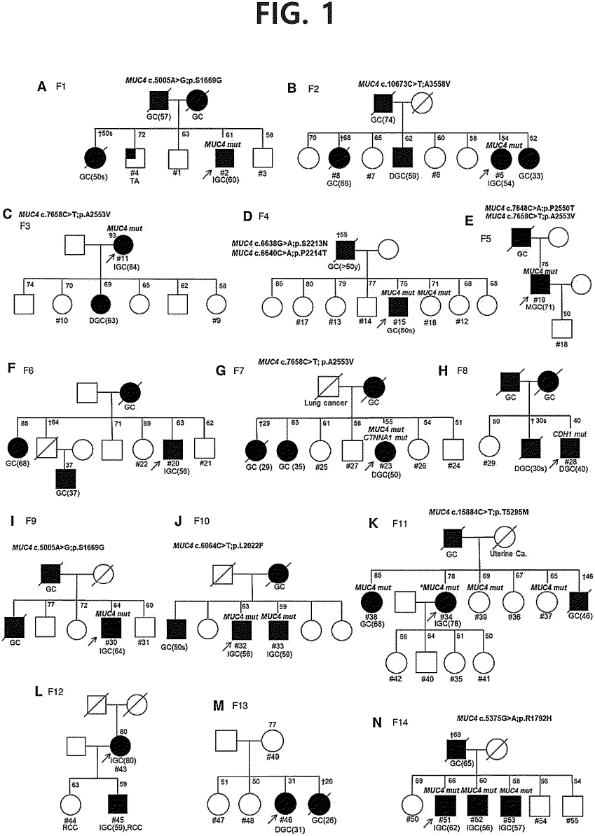

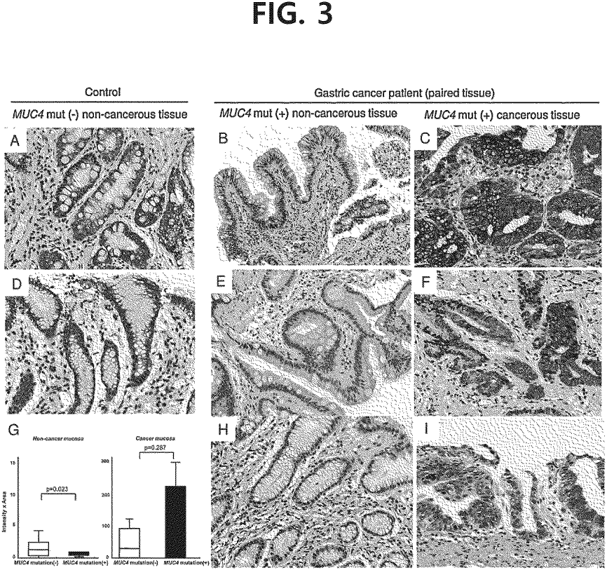

According to one embodiment of the present invention, MUC4-stained cells tended to decrease in noncancerous gastric mucosa, indicating that the normal gastric mucosa of subjects with MUC4 mutations had reduced MUC4 expression compared to subjects with normal MUC4 genes, and subjects with MUC4 mutations show reduced expression of MUC4, which suggests that MUC4 mutations suppress MUC4 expression in the normal gastric mucosa and cause detrimental effects. In addition, this trend was most prominent in family members with the c.5375G>A:p.R1792H mutation of MUC4 (Family No. 14 in FIG. 1), and MUC4 expression increased in cancer tissues compared to normal tissues. Through this, it was found that the excessive expression of MUC4 in cancer tissue showed that the MUC4 gene played a dual role as an oncogene (Example 4 and FIG. 3).

The MUC4 gene mutation according to one aspect of the present invention may be mutation in germline, specifically, mutation in the exon region of the MUC4 gene, and more specifically, the MUC4 gene mutation may be a mutation at one or more loci selected from the group consisting of exon 2 and exon 24 of the MUC4 gene.

The mutation at the rs547775645 locus on the MUC4 gene according to one aspect of the present invention may be a missense mutation.

The MUC4 gene mutation according to one aspect of the present invention may be one or more mutations selected from the group consisting of NM_018406.7:c.5375C>T, NM_018406.7:c.5005T>C, NM_018406.7:c.7658G>A, NM_018406.7:c.11180G>C, NM_018406.7:c.15884G>A, NM_018406.7:c.10673G>A, NM_018406.7:c.6064G>A, NM_018406.7:c.7648G>T, NM_018406.7:c.6638C>T, NM_018406.7:c.6640G>T, and NM_018406.7:c.3053G>C.

In one aspect of the present invention, it is characterized that MUC4 gene mutation is specifically detected in a gastric cancer subject compared to a normal control subject, and based on this, the MUC4 gene mutation is provided as a biomarker for predicting or diagnosing gastric cancer.

A detecting agent according to one aspect of the present invention may refer to a substance that can be used to detect the presence of a mutation in the MUC4 gene, which is a predictive or diagnostic marker of gastric cancer, in a sample. The agent may be one or more selected from the group consisting of antisense oligonucleotide, primer pair, probe, antibody, peptide, and polynucleotide that specifically binds to the mutation.

The composition according to one aspect of the present invention may be applied to a sample of a subject, and the sample refers to all samples obtained from an individual in which the expression of the biomarker according to one aspect of the present invention may be detected. Specifically, the sample may be one or more selected from the group consisting of saliva, biopsy, blood, skin tissue, liquid culture, feces and urine, but is not limited thereto, and may be treated and prepared by a method which is generally used in the art.

The detecting agent according to one aspect of the present invention may be one or more selected from the group consisting of antisense oligonucleotide, primer pair, probe, and polynucleotide that specifically binds to the mutation. That is, detection of a nucleic acid may be performed by an amplification reaction using one or more oligonucleotide primer that hybridizes to a nucleic acid molecule encoding a gene or a complement of the nucleic acid molecule. For example, the detection of a nucleic acid using a primer may be performed by amplifying a gene sequence using an amplification method such as PCR, and then confirming whether the gene is amplified by a method known in the art.

In one aspect of the present invention, the “primer” refers to a polynucleotide having a base sequence that can complementarily bind to the end of a specific region of a gene, or a mutant thereof, which is used for amplifying the specific region corresponding to a target region of the gene by PCR. The primer is not required to be completely complementary to the end of the specific region, and may be used as long as it is complementary to the end to such an extent that it can form a double-stranded structure by hybridizing with the end.

In one aspect of the present invention, the “probe” refers to a polynucleotide having a base sequence that can complementarily bind to a target of a gene, a mutant thereof, or a polynucleotide and a labeling substance bound thereto.

In one aspect of the present invention, “hybridization” means that 2 single-stranded nucleic acids form a duplex structure by pairing complementary base sequences.

Hybridization may occur not only when there is complete complementary pairing between single-stranded nucleic acid sequences (perfect match), but also when there are partially mismatched (mismatch) bases.

The detecting agent according to one aspect of the present invention may be one or more selected from the group consisting of antibody and peptide that specifically binds to the amino acid site of the mutation.

The MUC4 mutation according to one aspect of the present invention may be one or more amino acid mutations selected from the group consisting of p.Arg1792His, p.Ser1669Gly, p.Ala2553Val, p.Thr3727Ser, p.Thr5295Met, p.Ala3558Val, p.Leu2022Phe, p.Pro2550Thr, p.Ser2213Asn, p.Pro2214Thr and p.Ser1018Cys, and the detecting agent according to one aspect of the present invention may be the agent capable of detecting the amino acid mutation of the mutation.

In one aspect of the present invention, the antibody may be one or more selected from the group consisting of a polyclonal antibody, a monoclonal antibody, a recombinant antibody, and a combination thereof. Specifically, the antibody may include all of not only the polyclonal antibody, the monoclonal antibody, the recombinant antibody, and a complete form having two light chains with the full length and two heavy chains with the full length, but also functional fragments of the antibody molecules, for example, Fab, F(ab′), F(ab′)2, and Fv. The antibodies may be easily prepared by using a well-known technique in the art and antibodies which are prepared and commercially sold may be used.

The composition according to one aspect of the present invention may further comprise labels which may quantitatively or qualitatively measure formation of an antigen-antibody complex, general tools used in the immunological analysis, reagents, and the like as well as the agent for measuring the presence or expression of the MUC4 gene mutation.

In one aspect of the present invention, the labels which may quantitatively or qualitatively measure the formation of the antigen-antibody complex include enzymes, fluorescent substances, ligands, luminescent substances, microparticles, redox molecules, radioactive isotopes, and the like, and are not necessarily limited thereto. The enzymes usable as the detection label include β-glucuronidase, β-glucosidase, β-galactosidase, urease, peroxidase, alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, β-lactamase, and the like, and are not limited thereto. The fluorescent substances include fluorescein, isothiocyanate, rhodamine, phycoerythrin, phycocyanin, allophycocyanin, o-phthaldehyde, fluorescamine, and the like, and are not limited thereto. The ligands include biotin derivatives and the like, and are not limited thereto. The luminescent substances include acridinium ester, luciferin, luciferase, and the like, and are not limited thereto. The microparticles include colloidal gold, colored latex, and the like, and are not limited thereto. The redox molecules include ferrocene, ruthenium complex compounds, viologen, quinone, Ti ions, Cs ions, diimide, 1,4-benzoquinone, hydroquinone, K₄W(CN)⁸, [Os(bpy)₃]²⁺, [RU(bpy)₃]²⁺, [MO(CN)₈]⁴⁻, and the like, and are not limited thereto. The radioactive isotopes include ³H, ¹⁴C, ³²P, ³⁵S, ³⁶Cl, ⁵¹Cr, ⁵⁷Co, ³⁸Co, ⁵⁹Fe, ⁹⁰Y, ¹²⁵I, ¹³II, ¹⁸⁶Re, and the like, and are not limited thereto.

In one aspect of the present invention, an example of the tool or the reagent includes suitable carriers, solubilizing agents, detergents, buffering agents, stabilizers, and the like, but is not limited thereto. When the marker is the enzyme, a substance and a quencher which may measure the enzyme activity may be included. The carriers include a soluble carrier, and an insoluble carrier. An example of the soluble carrier includes a buffer solution that is physiologically acceptable and known in the art, for example, PBS, and an example of the insoluble carrier may include polystyrene, polyethylene, polypropylene, polyester, polyacrylonitrile, fluororesin, cross-linked dextran, polysaccharide, other papers, glass, metal, agarose, and a combination thereof.

Gastric cancer according to one aspect of the present invention may be one or more selected from the group consisting of diffuse-type gastric cancer, intestinal-type gastric cancer, and mixed-type gastric cancer, but if it is gastric cancer, the type is not limited. Conventionally, biomarkers for gastric cancer associated with family history were limited to diffuse-type gastric cancer or hereditary diffuse gastric cancer (HDGC), but the composition according to one aspect of the present invention can predict or diagnose the presence or absence of MUC4 gene mutation regardless of the type of gastric cancer, and thus, there is an excellent effect of predicting or diagnosing a wider range of types of gastric cancer.

The composition according to one aspect of the present invention may predict or diagnose gastric cancer in a subject suffering from one or more cancers selected from the group consisting of gastric adenocarcinoma (STAD), colorectal cancer (CRC), and uterine corpus endometrial cancer (UCEC), but is not limited thereto.

According to one aspect of the present invention, a subject may or may not have a family history of gastric cancer, and having the family history of gastric cancer may mean that there are two or more family members who currently have gastric cancer or who have had gastric cancer in the past within three generations.

In another aspect, one aspect of the present invention provides a kit for predicting or diagnosing gastric cancer, comprising a composition for predicting or diagnosing gastric cancer comprising the detecting agent of a mucin 4 (MUC4) gene mutation. Descriptions of the MUC4 gene, MUC4 gene mutation, detecting agent, gastric cancer, subject, sample, etc. are as described above.

A kit according to one aspect of the present invention may further comprise an instruction.

The instruction according to one aspect of the present invention may comprise a description for predicting or diagnosing gastric cancer when the mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on the MUC4 gene.

The kit according to one aspect of the present invention may be applied to a subject with or without a family history of gastric cancer, and having the family history of gastric cancer may mean that there are two or more family members who currently have gastric cancer or who have had gastric cancer in the past within 3 generations.

The kit according to one aspect of the present invention may be applied to a subject suffering from cancer, and specifically, a subject suffering from one or more cancers selected from a group consisting of stomach adenocarcinoma (STAD), colorectal cancer (CRC), and uterine corpus endometrial cancer (UCEC).

In still another aspect, the present invention provides a method for providing information for predicting or diagnosing gastric cancer comprising extracting genomic DNA from a sample of a subject; and detecting a mutation at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on a mucin 4 (MUC4) gene in the extracted genomic DNA. Descriptions of the MUC4 gene, MUC4 gene mutation, detecting agent, gastric cancer, etc. are as described above.

The detecting the mutation according to one aspect of the present invention may comprise detecting one or more amino acid mutations selected from the group consisting of p.Arg1792His, p.Ser1669Gly, p.Ala2553Val, p.Thr3727Ser, p.Thr5295Met, p.Ala3558Val, p.Leu2022Phe, p.Pro2550Thr, p.Ser2213Asn, p-Pro2214Thr and p.Ser1018Cys on the loci of the MUC4 gene. The detecting may detect the amino acid mutation using a detecting agent capable of detecting the amino acid mutation of the mutation according to one aspect of the present invention.

The detecting the mutation according to one aspect of the present invention may comprise reacting a primer specific to a contiguous nucleotide sequence selected from nucleotide sequences of one or more loci selected from the group consisting of NM_018406.7:c.5375C>T, NM_018406.7:c.5005T>C, NM_018406.7:c.7658G>A, NM_018406.7:c.11180G>C, NM_018406.7:c.15884G>A, NM_018406.7:c.10673G>A, NM_018406.7:c.6064G>A, NM_018406.7:c.7648G>T, NM_018406.7:c.6638C>T, NM_018406.7:c.6640G>T, and NM_018406.7:c.3053G>C of the MUC4 gene; and amplifying the reactant.

The detecting the mutation according to one aspect of the present invention may be performed by targeted molecule cloning and sequence analysis by using a well known technique in the art. For example, the detecting may performed using any one or more techniques selected from the group consisting of DNA sequence analysis; primer extending assay including allele-specific nucleotide mixing assay and allele-specific primer extending assay (for example, allele-specific PCR, allele-specific ligation chain reaction (LCR), and gap-LCR); allele-specific oligonucleotide hybridizing assay (for example, oligonucleotide ligation assay); a cleave protection assay that detects mismatched bases in a double strand of nucleic acid by using protection from a cleaver; MutS protein binding analysis; electrophoresis analysis comparing mobility of a variant and a wild type nucleic acid molecule; deformation-gradient gel electrophoresis (DGGE, for example, the same as the literature [Myers et al, (1985) Nature 313:495]); analysis of RNase cleavage in mismatched base pairs; analysis of chemical or enzymatic cleavage of a hetero double-stranded DNA; mass spectrometry (for example, MALDITOF); genetic bit analysis (GBA); 5′ nucleases assay (for example, TaqMan); and an assay using molecular beacon, but is not limited thereto.

The MUC4 gene mutation according to one aspect of the present invention may be one or more mutations selected from the group consisting of NM_018406.7:c.5375C>T, NM_018406.7:c.5005T>C, NM_018406.7:c.7658G>A, NM_018406.7:c.11180G>C, NM_018406.7:c.15884G>A, NM_018406.7:c.10673G>A, NM_018406.7:c.6064G>A, NM_018406.7:c.7648G>T, NM_018406.7:c.6638C>T, NM_018406.7:c.6640G>T, and NM_018406.7:c.3053G>C.

The method for providing information according to an aspect of the present invention may be one for predicting or diagnosing gastric cancer when the MUC4 gene mutation is detected after detecting the MUC4 gene mutation.

The method for providing information according to one aspect of the present invention may be one for predicting or diagnosing one or more selected from the group consisting of diffuse-type gastric cancer, intestinal-type gastric cancer, and mixed-type gastric cancer, but if it is gastric cancer, the type is not limited. Conventionally, biomarkers for gastric cancer associated with family history were limited to diffuse-type gastric cancer or hereditary diffuse gastric cancer (HDGC), but the method for providing information according to one aspect of the present invention can predict or diagnose the presence or absence of MUC4 gene mutation regardless of the type of gastric cancer, and thus, there is an excellent effect of predicting or diagnosing a wider range of types of gastric cancer.

According to one aspect of the present invention, a subject may or may not have a family history of gastric cancer, and having the family history of gastric cancer may mean that there are two or more family members who currently have gastric cancer or who have had gastric cancer in the past within three generations.

The subject according to one aspect of the present invention may be a subject suffering from cancer, specifically, a subject suffering from one or more cancers selected from the group consisting of stomach adenocarcinoma (STAD), colorectal cancer (CRC), and uterine corpus endometrial cancer (UCEC).

Hereinafter, the configuration and effects of the present invention will be described in more detail by way of examples. However, the following examples are for illustrative purposes only and it will be apparent to those of ordinary skill in the art that the scope of the present invention is not limited by the examples.

### [Example 1] Characteristics of Subjects

To identify novel gastric cancer-susceptibility genes, whole-exome sequencing (WES) was performed on 19 gastric cancer patients from 14 families in which 2 or more gastric cancer cases occurred within 3 generations and 36 first-degree relatives who did not have gastric cancer. For this purpose, the subject enrollment method and the characteristics of the enrolled subjects are as follows.

### Patient Enrollment for Exome Sequencing

From April 2017 to March 2018, gastric cancer patients and their first-degree relatives (FDRs) with two or more family members diagnosed with gastric cancer within three generations among the families at Seoul National University Bundang Hospital were enrolled in the study according to one example of the present invention. A non-gastric cancer control (hereafter referred to as a control) was defined as a person over 50 years of age who had undergone a normal endoscopy within the past 6 months. The diagnosis of gastric cancer was based on pathological diagnosis by endoscopic biopsy or surgical specimen.

Family history of gastric cancer, smoking, drinking, dietary preference, socioeconomic status, gastrointestinal symptoms, and previous Helicobacter pylori eradication history were obtained through a questionnaire. Histological evaluation by Giemsa staining and anti-Helicobacter pylori test was performed to confirm the Helicobacter pylori infection status. [Choi YJ, Kim N, Jang W, Seo B, Oh S, Shin CM, et al. Familial Clustering of Gastric Cancer: A Retrospective Study Based on the Number of First-Degree Relatives. Medicine (Baltimore). 2016; 95(20): e3606. Epub 2016/05/20. https://doi.org/10.1097/MD.0000000000003606 PMID: 27196462; PubMed Central PMCID: PMC4902404.][Kim N, Cho SI, Yim JY, Kim JM, Lee DH, Park JH, et al. The effects of genetic polymorphisms of IL-1 and TNF-A on Helicobacter pylori-induced gastroduodenal diseases in Korea. Helicobacter. 2006; 11(2):105-12. Epub 2006/04/04. https://doi.org/10.1111/j.1523-5378.2006.00384.x PMID: 16579840].

All procedures involving subjects were performed in accordance with the ethical standards of institutional and national research committees and the 1964 Declaration of Helsinki. This study was approved by the Institutional Review Board of Seoul National University Bundang Hospital (B-1610-366-303). All family members participating in this study signed a specific informed consent form.

### Characteristics of Subjects

Total subjects included 55 subjects (19 gastric cancer patients and 36 non-gastric cancer relatives) from 14 independent families. The family tree of 14 families is shown in FIG. 1. Three HDGC (hereditary diffuse gastric cancer) families (Nos. 7, 8 and 13) who met the International Gastric Cancer Linkage Consortium 2010 clinical criteria were included. [Fitzgerald RC, Hardwick R, Huntsman D, Carneiro F, Guilford P, Blair V, et al. Hereditary diffuse gastric cancer: updated consensus guidelines for clinical management and directions for future research. J Med Genet. 2010; 47(7):436-44. Epub 2010/07/02. https://doi.org/10.1136/jmg.2009.074237 PMID: 20591882; PubMed Central PMCID: PMC2991043]. The clinical characteristics of the subjects were shown in Table 1 below (Table 1).

TABLE 1

Clinical and demographic characteristics of gastric cancer patients and non-gastric cancer subjects

| Catagory | Non-gastric cancer (=36) | Gastric cancer (=19) | P-value^{a} |
|---|---|---|---|
| Male | 12 (33.3) | 12 () | 0.034 |
| Age (years) | 62.14 (9.1) | 55.11 (13.8) | 0.331 |
| MUC1 (rs4072037) | | | |
| GS | 0 | 2 (10.5) | 0.098 |
| AG | 5 (13.9) | 1 (5.3) | |
| AG | 31 (96.1) | 16 (84.2) | |
| Local residence | 21 (58.3) | 14 (73.7) | 0.260 |
| Smoking | 10 (27.8) | 12 (63.2) | 0.011 |
| Drinking | 22 (61.1) | 14 (73.7) | (0.351) |
| Frequency of fruit intake ≥ 3/week | 26 (72.2) | 16 (84.2) | 0.320 |
| H. pylori | 27 (75.0) | 11 (57.9) | 0.192 |
| Blood type with the 8 allete | 11 (30.6) | 6 (31.6) | 0.938 |
| MUC4 mutation | 3 (8.3) | 14 (73.7) | <0.001 |
| histology of cancer | | | |
| intestinal-type | | 13 (63.2) | |
| Diffuse-type | | 4 (26.3) | |
| Unknown | | 2 (10.5) | |
| HDGC⁵ | | 3 (15.8) | |

Most values are expressed as numbers (%) except for age, which is expressed as the mean (standard deviation).
Bold letters indicate statistical significance.
Abbreviations are as follows gastric cancer (GO) Hereditary diffuse gastric cancer syndrome (HDGC).
^{a} Statistical significance determained by chi-squared test or t-test.
^{b} classification criteria
^{c} including 1 mixed-type
^{d} Family with 2 or more cases of gastric cancer with at least 1 diffuse-type gastric cancer diagnosed before age 50

As shown in Table 1, the average age of patients diagnosed with gastric cancer was 59.0 years (range: 31 to 84 years), whereas the average age of relatives without gastric cancer was 62 years. There was a tendency for a higher proportion of males in the gastric cancer group than that in the other groups (63.2% in the gastric cancer group vs. 33.3% in the non-gastric cancer group, p = 0.034). The smoking rate was higher in gastric cancer patients than that in the non-gastric cancer group (63.2% in the gastric cancer group vs. 27.8% in the non-gastric cancer group, p = 0.011). About half of gastric cancer patients were H. pylori-positive, and 75.0% of their non-cancerous relatives were H. pylori-positive, with no significant difference in these rates. According to the Lauren classification, among the enrolled gastric cancer patients, 3 were identified as diffuse-type, 13 as intestinal-type, and the rest as mixed type. Information on the specific histologic type of the remaining patients was requested from the hospital that had treated the patient for gastric surgery or histology long ago, but was unable to identify their type.

### [Example 2] Genomic Analysis of Subjects to Discover Gastric Cancer-Related Biomarkers

In order to discover biomarkers for predicting or diagnosing gastric cancer in the subjects of Example 1, the subject’s genome was analyzed as follows.

### DNA Isolation and Whole-Exome Sequencing (WES)

First, genomic DNA from the subjects of Example 1 was isolated using the Qiagen DNeasy blood and tissue kit (Qiagen, Hilden, Germany) according to the manufacturer’s instructions. To perform WES. Agilent SureSelect All Exon V6 (Agilent Technologies, Santa Clara, Calif.) along with reagents were used along with sequencing libraries and capture. Sequencing was performed on an Illumina HiSeq 2500 platform (2 × 100 bp-paired ends: Illumina, Inc., San Diego. CA). The sequence dataset of 55 enrollments of Example 1 was deposited in the European Nucleotide Archive (http://www.ebi.ac.uk/ena/data/view) under the accession number PRJEB29071.

### Mutation Detection and Annotation

Raw sequencing reads were aligned to Human Genome Reference Assembly GRCh37/hg19 using Burrows-Wheeler Aligner (BWA v0.7.15) software. [Li H, Durbin R. Fast and accurate short read alignment with Burrows-Wheeler transform. Bioinformatics. 2009; 25(14):1754-60. Epub 2009/05/20. https://doi.org/10.1093/bioinformatics/btp324 PMID: 19451168; PubMed Central PMCID: PMC2705234]. BWA alignment files were convened to BAM files using SAM tools v1.3 and copies were marked as Picard (https://sourceforge.net/projects/picard, v1.96). Local realignment, base quality recalibration, and haplotype calling were performed in genomic Variant Call Format (gVCF) mode for each sample from each subject using the Genome Analysis Toolkit (GATK v3.5) according to best case [DePristo MA, Banks E. Poplin R, Garimella KV, Maguire JR, Hartl C, et al. A framework for variation discovery and genotyping using next-generation DNA sequencing data. Nat Genet. 2011; 43(5):491-8. Epub 2011/04/12. https://doi.org/10.1038/ng.806 PMID: 21478889; PubMed Central PMCID: PMC3083463]. Genomic VCF (gVCF) files were combined and co-genotyped with GATK. Functional annotation of genetic mutations was performed using ANNOVAR with population frequencies. Mutations on Exon 24 of the MUC4 gene were identified by Sanger sequencing and all mutation reads were examined with the Integrative Genomes Viewer [Robinson JT, Thorvaldsdouir H, Winckler W. Guttman M, Lander ES, Getz G, et al. Integrative genomics viewer. Nat Biotechnol. 2011; 29(1):24-6. Epub 201 1/01/12. https://doi.org/10.1038/nbt.1754 PMID: 21221095; PubMed Central PMCID: PMC3346182].

### Analysis of Linkage and Association

Loci of disease susceptibility genes were identified using linkage analysis and gene-based association tests with Pedigree Variant Annotation, Analysis, and Search Tool (pVAAST) under an autosomal dominant genetic model and the maximum allowable prevalence of disease of 0.005. [Hu H, Roach JC, Coon H. Guthery SL, Voelkerding KV, Margraf RL, et al. A unified test of linkage analysis and rare-variant association for analysis of pedigree sequence data. Nat Biotechnol. 2014; 32(7):663-9. Epub 2014/05/20. https://doi.org/10.1038/nbt.2895 PMID: 24837662; PubMed Central PMCID: PMC4157619]. The p values of logarithm of odds (LOD) scores for the linomial likelihood based on the number of alleles in the case weighted by functional prediction and the controls and gene-based burden-type composite likelihood ratio test (CLRT) were calculated from 10⁶ substituted samples using a gene-drop method. In the linkage analysis, considering each family structure, the present inventors compared the mutations in 19 gastric cancer patients with 36 controls who did not develop gastric cancer in all 14 families. In the gene-based association test, the present inventors compared the whole exome allele counts of 19 gastric cancer patients with the total genome allele counts of 397 Korean controls obtained from the Korea National Biobank at the National Institute of Korea. A total of 19,491 genes were analyzed, and the Bonferroni-adjusted 0.05 level was 2.57×10⁻⁶. For MUC4. 10⁸ substituted samples were used because the p value of the CLRT score in 10⁶ substituted samples was 1.0 × 10⁻⁶.

### Allele Frequency Determination

The gnomAD database (http://gnomad.broadinstitute.org/) was used to determine the frequency of specific mutations in the total and East Asian control populations.

### The Cancer Genome Atlas (TCGA) Data Analysis

TCGA data was downloaded from the Genomic Data Commons Legacy (GRCh37/hg19) archive and the Institute for Systems Biology Cancer Genomics Cloud at the Genomic Data Commons Data Portal (https://portal.gdc.cancer.gov). Sequence information was obtained from database of genotypes and phenotypes (dbGaP).

### Effect Size Analysis

The odds ratio (OR) for all significant genes at the Bonferroni-adjusted 0.05 significance level was estimated by logistic regression. Family relationships were estimated with GMMAT [Chen H, Wang C. Conomos MP, Stilp AM, Li Z, Sofer T. et al. Control for Population Structure and Relatedness for Binary Traits in Genetic Association Studies via Logistic Mixed Models. Am J Hum Genet. 2016; 98(4):653-66. Epub 2016/03/29. https://doi.org/10.1016/j.ajhg.2016.02.012 PMID:27018471; PubMed Central PMCID: PMC4833218], and the variance of random effects explaining family relationships was estimated to be zero. Therefore, gastric cancer status among family members was assumed to be independent, and standard logistic regression analysis was applied using Rex Version 2.1 (http://rexsoft.org). For each gene, if one or more rare alleles were observed in the corresponding gene, the genetic risk score was coded as 1, and otherwise coded as 0. Gender, age, smoking status, and HDGC were included as covariates to adjust for effects.

### Discovery of Germline Exon Mutation Associated With Gastric Cancer

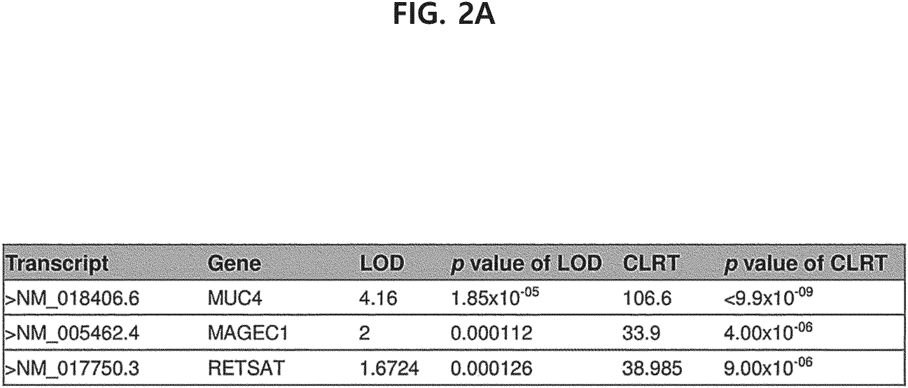

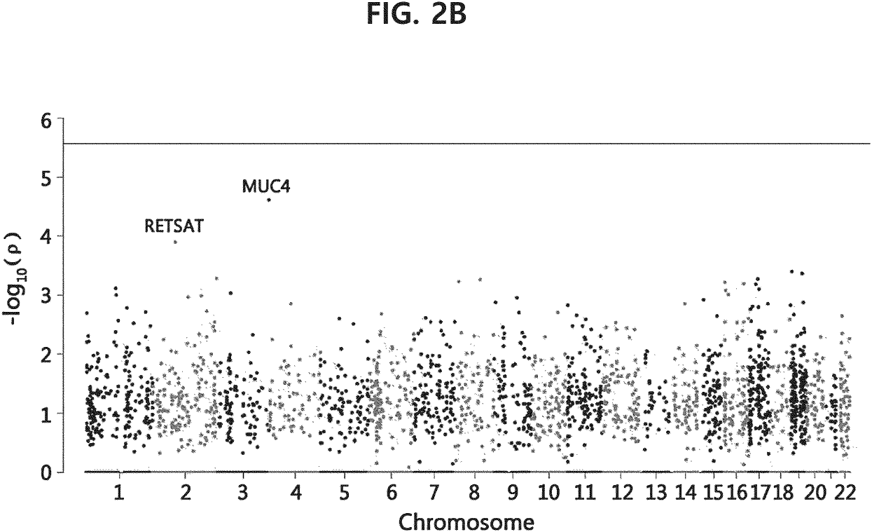

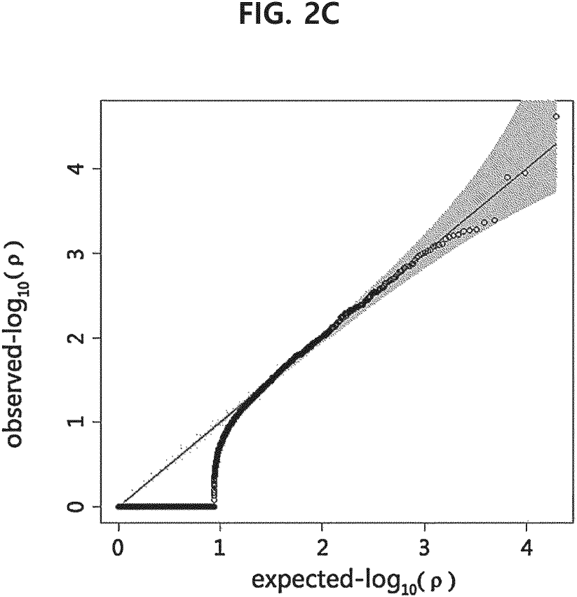

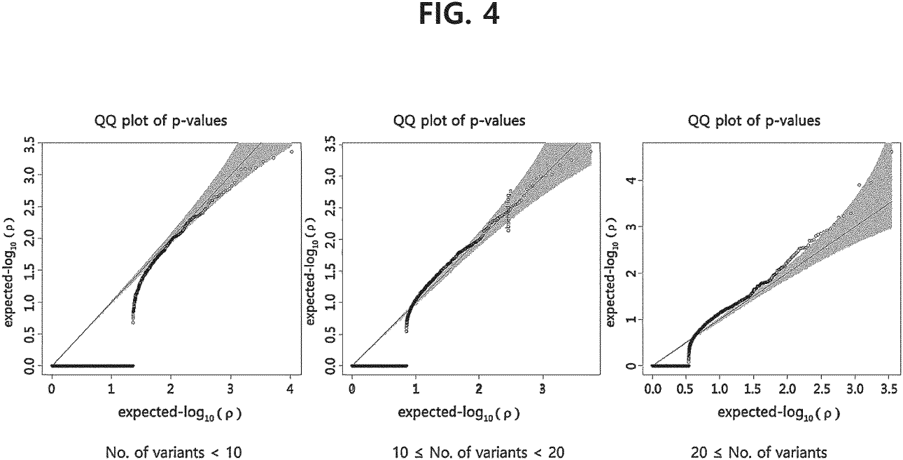

The WES data obtained from the gene analysis was generated from 55 subjects with a mean depth of 96 times in the target exome region. An average of 97% of all target regions were at least 20-fold. To search for rare mutation candidates for gastric cancer, the linkage analysis combined with association test was performed. Based on the LOD p value. MUC4, MAGEC1, and RETSAT were identified as putative genes related to gastric cancer, which were shown in FIGS. 2A to 2C. In the gene-based CLRT analysis incorporating linkage information, case-control associations, and functional variant prediction, MUC4 reached genome-wide significance levels (p value ≤ 9.9 × 10⁻⁹, and genome-wide Bonferroni-adjusted 0.05 significance level = 2.6 × 10⁻⁶) (FIG. 2A). FIGS. 2B and 2C show the Manhattan and quantile-quantile (QQ) plots for this linkage analysis, respectively, and show that this statistical analysis maintains the nominal significance level. FIG. 4 shows QQ plots of LOD p values of three different gene size groups, confirming that the analysis was not affected by gene size.

### MUC4 Mutations

The entire dataset was analyzed using the above pVAAST, and 14 MUC4 mutations were found to contribute to the LOD score, of which 10 mutations were finally selected with LOD values greater than 0. Ten MUC4 mutations were identified among 14 independent families (Table 2).

TABLE 2

Characteristics of germline MUC4 mutations associated with gastric cancer derived by linkage analysis in the 14 families studied

| Location^{a} | Allelic change | | Amino Acid change | LOD | Outbreak case | Collective MAF | Exon | Function prediction |
|---|---|---|---|---|---|---|---|---|
| | Ref | Alt | (NM_018406) | | (Patient IDs)^{b} | (All/East Asian. %)^{c} | | |
| chr395513076 | C | T | pArg1792 | 0.60 | 51, 52, 53 | 0.044/0.453 | 2 | O-glycosylation |
| rs774527434 | | | | | | | | (alpha subunit) |
| chr3195513446 | T | C | p.Ser1669Gly | 1.20 | 34, 2, 30 | 0.262/1.450 | 2 | O-glycosylation |
| rs534579185 | | | | | | | | (alpha subunit) |
| chr395510793 | G | A | pAla2553Val | 1.57 | 11, 19, 23 | 0.062/0.023 | 2 | O-glycosylation |
| rs77250903 | | | | | | | | (alpha subunit) |
| chr3195507271 | G | C | p.The57275er | 0.50 | 51 | 0.017/0.226 | 2 | O-glycosylation |
| rs558957409 | | | | | | | | (alpha subunit) |
| chr3195475923 | G | A | p.The529SMet | 0.60 | 34, 37, 38, 39 | 0.001/0.011 | 24 | N-glycosylation |
| rs53195105 | | | | | | | | (beta subunit) |
| chr53507778 | G | A | p.Ala3558Val | 0.35 | 5 | 0.016/13.000 | 2 | O-glycosylation |
| rs754805153 | | | | | | | | (alpha subunit) |
| chr3195512387 | G | A | p.Lgu2022Pha | 0.30 | 32, 33 | 0.001/0.009 | 2 | O-glycosylation |
| rs1304612772 | | | | | | | | (alpha subunit) |
| chr3195510303 | G | T | [symbol] | 0.30 | 19 | 0.003/0.005 | 2 | O-glycosylation |
| rs774507241 | | | | | | | | (alpha subunit) |
| chr1195511313 | C | T | [symbol] | 0.14 | 15, 16 | 0.001/0.009 | 2 | O-glycosylation |
| rs771925912 | | | | | | | | (alpha subunit) |
| chr3155511311 | G | T | p.P2214T | 0.54 | 15, 16 | 5.006/0.500 | 2 | O-glycosylation |
| rs745342765 | | | | | | | | (alpha subunit) |

^{a} Chromosome in a reference genome,
^{b} 10s of individuals diagnosed with gastric cancer are highlighted in bold and underlined case numbers belong to the same family
^{c} MAP (96) in total and East Asian populations in the gnomAD (v2.3) exome database [symbol]
indicates text missing or illegible when filed

As shown in Table 2 above, all subjects with MUC4 mutations were gastric cancer patients except cases #16, #37, and #39. Most of the gastric cancer patients with MUC4 mutations were intestinal gastric cancer except for #23 (FIG. 1). All three subjects (#16, #37 and #39) who did not have gastric cancer were female and non-smokers. Two mutations, c.7658C>T p.A2553V and c.5005A>G p.S1669G, were identified in three unrelated families. Subjects #15, #16, #19, #34 and #51 each had two different mutations. Mutation identification rates in groups of East Asian origin were mostly higher than those observed in the entire population (Table 2).

On the other hand, among the subjects of Example 1, the frequency of the MUCI rs4072037 A allele was 90.9%, similar to that of 1,124 Chinese gastric cancer patients [Qiu LX, Hua RX, Cheng L, He J. Wang MY, Zhou F, et al. Genetic variant rs4072037 of MUC1 and gastric cancer risk in an Eastern Chinese population. Oncotarget. 2016; 7(13):15930-6. Epub 2016/02/26. https://doi.org/10.18632/oncotarget.7527 PMID: 26910281; PubMed Central PMCID: PMC4941287], while the frequencies of the A and G alleles were 57.9% and 42.1% in the American group and 49.2% and 50.8% in the African group, respectively [Reis CA, David L, Seixas M, Burchell J, Sobrinho-Simoes M. Expression of fully and under-glycosylated forms of MUC1 mucin in gastric carcinoma. Int J Cancer. 1998; 79(4):402-10. Epub 1998/08/12. https://doi.org/10.1002/(sici)1097-0215(19980821)79:4<402::aid-ijc16>3.0.co;2-6 PMID: 9699534]. These results indicate that East Asian populations may be genetically vulnerable through MUC1 mutation. Most of the frequencies of MUC4 mutations identified from the above analysis were genetically higher in the East Asian population than that in the global population including Western origin (Table 2). In addition, one type of mutation (rs774527434) in MUC4 was identified in East Asian gastric cancer patients in TCGA. Overall, MUC4 mutations putative in the present invention may contribute to geographic differences in gastric cancer incidence, parallel to MUC1 mutations.

In addition, since gastric cancer has a heterogeneous etiology, individuals in gastric cancer families may show discrepancies between genetic susceptibility and clinical expression. As a result of the genome analysis, 5 gastric cancer patients without MUC4 mutations were identified among 4 independent families: #20, #28, #43, #45, and #46 (FIG. 1). Patient #28 with diffuse-type gastric cancer who met HDGC criteria had a novel missense mutation in CDH1 (NM_001317184: exon8:c.G1057A:p.E353K). Although family #46 met the HDGC criteria, no mutations were found in CDH1 or CTNNA1 [Majewski IJ, Kluijt I, Cats A, Scerri TS, de Jong D, Kluin RJ, et al. An alpha-E-catenin (CTNNA1) mutation in hereditary diffuse gastric cancer. J Pathol. 2013; 229(4):621-9. Epub 2012/12/05. https://doi.org/10.1002/path.4152 PMID: 23208944][ Hansford S, Kaurah P, Li-Chang H, Woo M, Senz J, Pinheiro H, et al. Hereditary Diffuse Gastric Cancer Syndrome: CDH1 Mutations and Beyond. JAMA Oncol. 2015; 1(1):23-32. Epub 2015/07/17. https://doi.org/10.1001/jamaoncol.2014.168 PMID: 26182300]. Patients #43 and #45 belonged to the same family with two patients with renal cell carcinoma, suggesting the possibility of a different genetic syndrome. In particular, it was found that MUC4 mutation and the occurrence of gastric cancer can have a strong association when most of the gastric cancers are intestinal-type and not HDGC in Koreans.

### Effect Size of MUC4 Mutations in the Development of Gastric Cancer

Standard logistic regression analysis was applied to all 55 subjects by adjusting for gender, smoking, and HDGC, and the results are shown in Table 3 below.

TABLE 3

Odds ratios of independent risk factors for gastric cancer by binomial logistic regression analysis

| Category (reference) | log (OR) | OR | SE | 95% Cl of OR | Z-value | p-value |
|---|---|---|---|---|---|---|
| Gender [Female] | 1.27 | 3.56 | 1.05 | (0.45 to 27.63) | 1.21 | 0.225 |
| Any MUC4 mutation involved [N/A] | 4.1 | 56.58 | 1.06 | (7.33 to 459.37) | 3.85 | <0.001 |
| HDGC [N/A] | 1.02 | 2.78 | 1.04 | (0.36 to 21.36) | 0.95 | 0.326 |
| Smoking (Present/Past/[NA]) | 1.17 | 2.22 | 3.08 | (0.45 to 22.80) | 1.17 | 0.241 |

OR adds. ratio; SE standard error Cl, confidential interval;
HDGC, hereditary gastric cancer syndrome
MUC4 mutation, smoking, and family history of hereditary diffuse gastric cancer were adjusted for gender.
Pseudo R² = 0.616
Bold text indicates statistical significance.

As shown in Table 3 above, it was found that having any MUC4 mutation is associated with an increased risk of gastric cancer.

### [Example 3] Verification of Association Between MUC4 and Gastric Cancer in a Large Cohort

As a result of genomic analysis of the subjects of Example 1, it was confirmed that the risk of gastric cancer increased in subjects with MUC4 mutations from Example 2. Therefore, the association between the MUC4 mutation and gastric cancer was verified in a larger cohort in the following manner.

### Genome-Wide Association of Mutations Located in MUC4

Blood samples were collected from 597 histologically confirmed gastric cancer patients and 9,758 non-gastric cancer patients (controls) at Seoul National University Bundang Hospital and Seoul National University Hospital Health Care System Gangnam Center. The samples were genotyped by Affymetrix Axiom Korean Chip consisting of 827,783 mutations.

In this case, the following subjects were excluded: 1) if the sex estimated by the genome is different from the clinical information, 2) if the call rate of the subject is less than 97%, 3) if the heterozygosity rate is 3 times the standard deviation from the mean, 4) if the identity-by-descent (IBD) estimate with other subjects is greater than 0.185 and the missing rate is higher compared to its paired subjects.

In addition, the following mutations were excluded: 1) if the missing rate was greater than 3% or if there was a significant difference between gastric cancer patients and controls (p < 1 × 10⁻⁵), 2) if minor allele frequency is less than 5%, 3) if, as suggested in Anderson et al., the p value in the Hardy-Weinberg equilibrium precision test is less than 0.001 (Anderson CA, Pettersson FH, Clarke GM, Cardon LR, Morris AP, Zondervan KT. Data quality control in genetic case-control association studies. Nat Protoc. 2010; 5(9):1564-73. Epub 2010/11/19. https://doi.org/10.1038/nprot.2010.116 PMID: 21085122; PubMed Central PMCID: PMC3025522].

Then, the Michigan Imputation Server was used to impute untyped mutations [Das S, Forer L, Schonherr S, Sidore C, Locke AE, Kwong A, et al. Next-generation genotype imputation service and methods. Nat Genet. 2016; 48(10):1284-7. Epub 2016/08/30. https://doi.org/10.1038/ng. 3656 PMID: 27571263; PubMed Central PMCID: PMC5157836]. After imputation, 4,224 mutations located in MUC4 and their 0.5 MB flanking regions were adjusted for gender, age, and impacts of top 10 key factors of the sample relationship matrix and analyzed using logistic regression. The Bonferroni-adjusted 0.05 significance level was 1.18 × 10⁻⁵, and PLINK (v1.90b4.5) and R (v3.5.2) were used in the process. [Chang CC, Chow CC, Tellier LC, Vattikuti S, Purcell SM, Lee JJ. Second-generation PLINK: rising to the challenge of larger and richer datasets. Gigascience. 2015; 4:7. Epub 2015/02/28. https://doi.org/10.1186/s13742-015-0047-8 PMID: 25722852; PubMed Central PMCID: PMC4342193].

### Validation of Association Between MUC4 and Gastric Cancer in a Large Case-Control Cohort

Assuming that hereditary and sporadic gastric cancers may share a genetic background for gastric cancer, whether MUC4 mutations were associated with gastric cancer in a large cohort consisting of the above 597 gastric cancer patients and 9,759 healthy controls genotyped with SNP arrays was further analyzed. Common SNPs in the MUC4 region (chr3: 195,473,637-195,539,149) covering 0.5 MB of flanking regions were analyzed, with a Bonferroni-adjusted 0.05 significance level of 1.18×10⁻⁵. Two common mutations (rs148735556 and rs11717039) were detected in MUC4 lesions (Table 4), suggesting an association between MUC4 and gastric cancer.

TABLE 4

MUC4 area with 0.5 MB flanking area (chr3: 194,473,637-195,539,149)

| CHR | SNP | BP | P | OR | L95 | U95 | A1 | A2 | MAF_A | MAF_U | MAF_OR | HWE | MISS |
|---|---|---|---|---|---|---|---|---|---|---|---|---|---|
| 3 | rs148735556 | 195052426 | 1.27e-07 | 4.343 | 2.519 | 7.487 | T | A | 0.0235 | 0.0054 | 0.0113 | 0.3515 | 0 |
| 3 | rs11717039 | 195481737 | 1.071e-05 | 1.386 | 1.198 | 1.603 | C | T | 0.4782 | 0.4026 | 0.4534 | 0.07002 | 0 |

In the exon 2 and exon 24 regions of MUC4, there were 25 SNPs, and the rs547775645 missense mutation in exon 2 was identified as significant at the 0.05/25 = 2 × 10⁻³ significance level (Table 5).

TABLE 5

MUC4 region (exon 2 and exon 24)

| Exon | SNP | BP | AA change | Annotation | F | OR | L35 | U95 | A1 | A2 | MAF_A | MAF_U | HWE | MISS |
|---|---|---|---|---|---|---|---|---|---|---|---|---|---|---|
| 2 | c547775645 | 195515398 | | issingm | 1.12e-0.3 | 17.75 | 3.146 | 100.1 | C | 6 | 0.0080 | 0.004 | 1 | 0 |

0.05/25 = 2 × 10⁻³

The imputation quality of the 10 rare mutations mentioned above in MUC4 was poor (INFO < 0.5) and could not be tested in SNP chip analysis. However, MUC4 missense mutations were identified as a predisposing factor for familial aggregation of gastric cancer, and it was confirmed that a common mutation existed in MUC4 that had a significant association with gastric cancer.

That is, a common mutation in the MUC4 region significantly associated with gastric cancer was found in a large case-control cohort of gastric cancer, indicating that MUC4 and gastric cancer were associated.

### Frequency of MUC4 Germline Mutations in Patients With Various Cancer Types

Since MUC4 is expressed not only in the stomach but also in other tissues such as the colon, esophagus, small intestine, uterus and lung, and abnormal MUC4 expression has been reported in various types of carcinomas, including carcinomas of the lung, breast, pancreas, and stomach [Chaturvedi P, Singh AP, Batra SK. Structure, evolution, and biology of the MUC4 mucin. FASEB J. 2008; 22(4):966-81. Epub 2007/11/21. https://doi.org/10.1096/fj.07-9673rev PMID: 18024835; PubMed Central PMCID: PMC2835492], allele frequencies of the MUC4 mutation have been investigated in germline samples from patients with various cancer types, and it was confirmed that patients with germline MUC4 mutations have a higher risk of developing various types of cancer. In addition, using the germline mutations obtained from the blood of the TCGA data of Example 2, it was tested whether 10 rare mutations in the MUC4 gene were cancer-related, and it was confirmed that gastric cancer and 4 cancer types were associated with three rare types of MUC4 mutations (Table 6).

TABLE 6

Frequency of three SNPs in various cancer types from TCGA

| | STAD | CRC | UCEC | UUAD | UUSC |
|---|---|---|---|---|---|
| rs774527434 | 0.17% | 0% | 0% | 0% | 0% |
| rs534779185 | 0% | 4% | 0.56% | 0% | 0% |
| rs77250903 | 0% | 0.1% | 0% | 0% | 0% |

STAD, stomach adenocarcinomas;
CRC, colorectal cancer;
UCEC, uterine corpus endometrial cancer;
LUAD, lung adenocarcinoma;
LUSC, lung squamous cell cancer

Among the 10 mutations of MUC4 gene associated with familial gastric cancer shown in Table 2 above, a heterozygous rs774527434 SNP was identified in one patient (0.17%) out of 295 stomach adenocarcinoma germline samples (Table 6), which was about 4 times higher than that in the general population (0.04%, Table 2). Two mutations, rs534779185 and rs77250903, were identified in 372 colorectal cancer (CRC) patients, with frequencies of 4.0% and 0.13%, respectively, which were higher than those in the general population (0.26% and 0.06%, respectively, in Table 2). One mutation, rs534779185, was found in 265 uterine corpus endometrial cancer samples with a frequency of 0.56%. Ten mutations of the MUC4 gene were not identified in 408 lung squamous cell cancer patients and 495 lung adenocarcinoma patients. Through this, it was found that MUC4 mutations can be associated with gastrointestinal or genitourinary tract cancer.

### [Example 4] Confirmation of Functional Effects in Gastric Tissue Through Immunohistochemistry (IHC) Analysis

As it was confirmed through Examples 2 and 3 that the risk of gastric cancer increased in the subjects with MUC4 mutations, an immunohistochemical (IHC) analysis was performed as follows to determine what kind of functional effect MUC4 mutations actually have in gastric tissue.

### Immunohistochemical Analysis of Non-cancerous Gastric Mucosa and Gastric Cancer Tissue

The antral noncancerous mucosa was evaluated using IHC from 15 gastric cancer patients and 8 non-gastric cancer patients who consented for endoscopic biopsy. In the case of gastric cancer patients, cancer tissues were also stained. An antibody for detecting MUC4 (clone: 8G7) (1:100 dilution, Zeta Corporation, Arcadia, CA, USA) was used for IHC. The antibody used for the IHC is to detect the MUC4α region. The specificity of the antibody has been demonstrated by previous studies. Whole staining of sections (4 µm thick) was performed via the BenchMark XT Staining system and ultraVIEW Universal DAB Detection Kit (Ventana Medical Systems, Inc., Tucson, AZ, USA). MUC4 expression was evaluated using scientific microscopy as an area-dependent intensity multiplication (%), where staining was observed in epithelial glands as follows (0 to 300): 0 if no staining; 1+ if faint/barely perceptible partial staining; 2+ for weak to moderate staining; 3+ for strong staining. In cancer tissues, areas that were strongly stained were scored. Each sample was scored in a blinded manner by a single pathologist (Hye-Seung Lee).

### MUC4 Expression in Gastric Tissue From Subjects With MUC4 Mutations

The results of IHC analysis of MUC4 expression in the gastric mucosa performed to investigate the functional effects of the identified mutations are as follows.

First, representative immunochemical results of 5 subjects of Family No. 14 with a complete coset with the MUC4 mutation (rs774527434) and containing the largest number of gastric cancer patients were shown in FIG. 3. MUC4 mutation-negative non-cancerous gastric mucosa (#50, #54) (FIG. 3A and FIG. 3D) showed high intensity, whereas IHC results of MUC4 mutation-positive noncancerous mucosa of three patients (#51, #52 and #53) with MUC4 mutation were weak or negative. (FIG. 3B, FIG. 3E and FIG. 3H). In contrast, the cancer tissues from three gastric cancer patients (#51, #52 and #53) showed high IHC scores (FIG. 3C, FIG. 3F and FIG. 3I).

In general, noncancerous mucosa with MUC4 mutations show lower MUC4-positive staining scores than that of noncancerous mucosa with wild type (FIG. 3G; median [interquartile range]: 0 [10.0 to 30.0] vs. 70 [9.5 to 165.0], p = 0.023). In cancer tissues, the IHC staining tends to be more prominent in tissues with MUC4 mutations compared to wild-type (FIG. 3G; median [interquartile range]: (75.0 (0 to 240.0) vs. 30 (0 to 105.0), p = 0.287).

According to the results of the IHC analysis, MUC4-stained cells tended to decrease in non-cancerous gastric mucosa, which means that MUC4 expression was reduced in normal gastric mucosa of subjects with MUC4 mutation compared to subjects with wild type. MUC4 is structurally similar to MUC1, and the subjects with MUC4 mutations show reduced expression of MUC4, suggesting that MUC4 mutations suppress MUC4 expression in the normal gastric mucosa, causing detrimental effects. This trend was most pronounced in family members with the c.5375G>A:p.R1792H mutation in MUC4 (Family No. 14). In addition, MUC4 expression was increased in cancer tissues compared to normal tissues. Through which it was confirmed that excessive expression of MUC4 in cancer tissues causes the MUC4 gene to play a dual role as an oncogene.

### [Example 5] Prediction of MUC4 Protein Structure

As it was confirmed through Examples 2 to 4 that MUC4 mutation was a biomarker for predicting or diagnosing gastric cancer, computer analysis was performed as follows to predict the structure of the MUC4 protein.

### Computer Analysis of MUC4 Structure

Motif search, peptide cleavage, glycosylation and the prediction of protein structure were performing using protein sequence (refSeqID:NP_001191215) and mRNA sequence (refSeqID:NM_018406.6) obtained from the NCBI reference sequence database for MUC4 structural analysis [O′Leary NA, Wright MW, Brister JR, Ciufo S, Haddad D, McVeigh R, et al. Reference sequence (RefSeq) database at NCBI: current status, taxonomic expansion, and functional annotation. Nucleic Acids Res. 2016; 44(D1):D733-45. Epub 2015/11/11. https://doi.org/10.1093/nar/gkv1189 PMID:26553804; PubMed Central PMCID: PMC4702849]. Motif search was performed using the MotifFinder tool of GenomeNet (https://www.genome.jp). PeptideCutter was performed on the protein reference sequence (NP_001191215) and the MUC4a region of mutation sequence [Wilkins MR, Gasteiger E, Bairoch A, Sanchez JC, Williams KL, Appel RD, et al. Protein identification and analysis tools in the ExPASy server. Methods Mol Biol. 1999; 112:531-52. Epub 1999/02/23. https://doi.org/10.1385/1-59259-584-7:531 PMID: 10027275] to perform peptide cleavage prediction. NetOGlyc [Steentoft C, Vakhrushev SY, Joshi HJ, Kong Y, Vester-Christensen MB, Schjoldager KT, et al. Precision mapping of the human O-GalNAc glycoproteome through SimpleCell technology. EMBO J. 2013; 32(10):1478-88. Epub 2013/04/16. https://doi.org/10.1038/emboj.2013.79 PMID: 23584533; PubMed Central PMCID: PMC3655468]. Further, NetNGlyc (http://www.cbs.dtu.dk/services/NetNGlyc/) was used to predict O-GalNAc (N-acetylgalactosamine) and location of N-GalNAc modifications, respectively. MODELLER [Eswar N, Webb B, Marti-Renom MA, Madhusudhan MS, Eramian D, Shen MY, et al. Comparative protein structure modeling using Modeller. Curr Protoc Bioinformatics. 2006;Chapter 5:Unit-5 6. Epub 2008104123. https://doi.org/10.1002/0471250953.bi0506s15 PMID: 18428767; PubMed Central PMCID: PMC4186674][Waterhouse A, Bertoni M, Bienert S, Studer G, Tauriello G, Gumienny R, et al. SWISS-MODEL: homology modelling of protein structures and complexes. Nucleic Acids Res. 2018; 46(Wl):W296-W303. Epub 2018/05/23. https://doi.org/10.1093/nar/gky427 PMID: 29788355; PubMed Central PMCID: PMC6030848]. Further, the protein structure was predicted using the homology modeling of SWISS-MODEL.

### Results of Protein Structure Prediction

Among the 10 MUC4 mutations identified in Examples 2 to 4, 9 mutations were located in exon 2, which contained a tandem repeat region [Chaturvedi P, Singh AP, Batra SK. Structure, evolution, and biology of the MUC4 mucin. FASEB J. 2008; 22(4):966-81. Epub 2007/11/21. https://doi.org/10.1096/fj.07-9673rev PMID: 18024835;PubMed Central PMCID: PMC2835492], and the other mutation was located in exon 24. However, homology modeling or first (ab initio) structural modeling was not successful due to the established MUC1 or MUC4 models and the absence of coil structures of O-glycosylation-rich sites.

According to glycosylation predictions [Steentoft C, Vakhrushev SY, Joshi HJ, Kong Y, Vester-Christensen MB, Schjoldager KT, et al. Precision mapping of the human O-GalNAc glycoproteome through SimpleCell technology. EMBO J. 2013; 32(10):1478-88. Epub 2013/04/16. https://doi.org/10.1038/emboj.2013.79 PMID: 23584533; PubMed Central PMCID: PMC3655468][ Gupta R, Brunak S. Prediction of glycosylation across the human proteome and the correlation to protein function. Pac Symp Biocomput. 2002:310-22. Epub 2002/04/04. PMID: 11928486], most MUC4 mutations in exon 2 are at or physically close to O-glycosylation sites (Table 7).

TABLE 7

Prediction of glycosylation sites using NetOGlyc and NetNGlyc

| | | |
|---|---|---|
| Location° | Amino acid change | Site Prediction |
| chr3395572387 | p.12022F | Locations (2020, 2021, 2023, 2024) are predicted to be O-glycolation sites |
| chr3:195570783 | pA2553V | Locations 0251, 2553, 2554, 2555) are predicted to be C-glycosylation sited |
| chr3155475523 | p.T5295A1 | Near or part of the last N-glycosylation site (10–Xaa-ST->ML) between the second and third EGF domains of the MUC4 B-subunit among about 20 predicted N-glcosylation sites |
| chr3055507271 | p.T37275 | Location (3727) is predicted to be O-glycosylation sites |
| chr30195507778 | p.A35558 | Location (356, 3559, 3550) are predicted to be O-glycosylation sites |
| chr195513078 | p.R1732B | Location (1790, 1791, 1 9 are predicted to be O-glycosylation sites |
| chr3195513445 | p.51889G | Locations (1858, 1855, 1870-2) are predicted to be O-glycosylation sites |
| chr3:195510803 | p.525507 | Locations (2548, 2547, \| ) 2551. 2552) are predicted to be O-glycosylation sites |
| chr3:1955171811 | p.P2214T | Locations (2212, 2218, \| } 2215, 2216) are predicted to be O-glycosylation sites |
| chr339551813 | p.52513N | Locations (2212, 2213, 2218, 2216)are predicted to be O-glycosylation sites |

^{<}Chromosome location in the reference genome GRCh37/hg19,
| ) indicates positive O-glycosylation sites

In particular, p.T3727S, p.S1669G and p.S2213N were more likely to have O-GalNAc deformations [Steentoft C, Vakhrushev SY, Joshi HJ, Kong Y, Vester-Christensen MB, Schjoldager KT, et al. Precision mapping of the human O-GalNAc glycoproteome through SimpleCell technology. EMBO J. 2013; 32(10):1478-88. Epub 2013/04/16. https://doi.org/10.1038/emboj.2013.79 PMID: 23584533; PubMed Central PMCID: PMC3655468]. A single codon change from G to A in this putative cleavage site (c.5375G>A:p.R1792H) could potentially inhibit proteolytic activity. A single codon change from C to T (c.15884C>T) resulted in interfering with threonine synthesis of a potential N-glycosylation site between the second and third epidermal growth factor (EGF)-like domains of the MUC4 ß subunit.

That is, the information obtained from in silico prediction of glycosylation in the MUC4 structure indicates that most regions encoded by MUC4 mutations are likely to be O-glycosylation sites. O-glycosylation with glycan micro-heterogeneity is important for the structure and function of mucin. Mucin-type glycans are involved in specific ligand-receptor interactions, can impart hygroscopicity, bind to various small molecules and proteins, and finally stabilize protein structures. [Jayaprakash NG, Surolia A. Role of glycosylation in nucleating protein folding and stability. Biochem J. 2017; 474(14):2333-47. Epub 2017/07/05. https://doi.org/10.1042/BCJ20170111 PMID: 28673927]. Although there are hundreds of O-glycosylation sites in the MUC4α subunit, a single amino acid difference at one specific site can alter the function of the encoded variant protein. [van der Post S, Thomsson KA, Hansson GC. Multiple enzyme approach for the characterization of glycan modifications on the C-terminus of the intestinal MUC2mucin. J Proteome Res. 2014; 13(12):6013-23. Epub 2014/11/19. https://doi.org/10.1021/pr500874f PMID: 25406038; PubMed Central PMCID:PMC4261943]. Glycosylation of MUC4 may have been altered based on the above preference for amino acid locations around the O-glycosylation site [Thanka Christlet TH, Veluraja K. Database analysis of O-glycosylation sites in proteins. Biophys J. 2001; 80(2):952-60. Epub 2001/02/13. https://doi.org/10.1016/s0006-3495(01)76074-2 PMID: 11159462; PubMed Central PMCID: PMC1301293] and changes in serine or threonine [Chaturvedi P, Singh AP, Batra SK. Structure, evolution, and biology of the MUC4 mucin. FASEB J. 2008; 22(4):966-81. Epub 2007/11/21. https://doi.org/10.1096/fj.07-9673rev PMID: 18024835; PubMed Central PMCID: PMC2835492].

On the other hand, previous studies have suggested that MUC4 can activate the ErbB2 oncoprotein during the pathogenesis of gastric cancer. [Yokoyama A, Shi BH, Kawai T, Konishi H, Andoh R, Tachikawa H, et al. MUC4 is required for activation of ErbB2 in signet ring carcinoma cell lines. Biochem Biophys Res Commun. 2007; 355(1):200-3. Epub 2007/02/13. https://doi.org/10.1016/j.bbrc.2007.01.133 PMID: 17292332] [Senapati S, Chaturvedi P, Sharma P, Venkatraman G, Meza JL, El-Rifai W, et al. Deregulation of MUC4 in gastric adenocarcinoma: potential pathobiological implication in poorly differentiated non-signet ring cell type gastric cancer. Br J Cancer. 2008; 99(6):949-56. Epub 2008/09/11. https://doi.org/10.1038/sj.bjc.6604632 PMID: 18781152; PubMed Central PMCID: PMC253875247, 48]. In addition, the mutation in exon 24, p.Thr5295Met, may be involved in ErbB2 signaling, as the mutation causes the amino acid change of the N-glycosylation site between the EGF-like domains.

Summarizing the above analysis results, the structural model of the putative third EGF-like domain of MUC4 spans residues F5300 to L5362, which is very close to the mutated residue T5295M. Since this site is only 5 residues away from the modeled EGF-like domain, it appears that this mutation could affect the function of the EGF-like domain.

### [Example 6] Verification of Association Between MUC4 Mutation and Gastric Cancer

As it was confirmed from Examples 2 to 4 that the risk of gastric cancer increased in subjects with MUC4 mutations, the association between the MUC4 mutation and gastric cancer was re-verified by analyzing genotyping and immunohistochemistry (IHC) in the following manner.

First, a total of 288 patients were selected from patients at Seoul National University Bundang Hospital, and among them, 237 subjects (103 gastric cancer patients and 134 non-gastric cancer patients (controls)) who completed immunohistochemistry (IHC) analysis of the gastric antrum and gastric cancer tissue was selected. The gastric antrum of the 237 selected subjects and blood and gastric cancer tissues in the case of gastric cancer patients were obtained, and genomic DNA was isolated and extracted from them (Qiagen DNeasy blood and tissue kit (Qiagen, Hilden, Germany)).

Using the extracted DNA, genotyping was performed on two SNPs of MUC4 rs774527434 and rs531395109. As a result, a total of 14 subjects had mutations in the MUC4 gene, of which 5 were non-gastric cancer patients and 9 were gastric cancer patients.

For reference, the primers and reporter sequences used for genotyping are as follows.

### MUC4 Rs774527434 SNP Measurement

- forward primer: CTTTCTTCAGCTTCCACAGATGAC (SEQ I
D NO: 1)

- reverse primer: TGGATGCCGAGGAAACGT (SEQ ID NO:
2)

- reporter 1: ACCACCCGTCTTCCT (SEQ ID NO: 3)

- reporter 2: ACCACCCATCTTCCT (SEQ ID NO: 4)

### MUC4 Rs531395109 SNP Measurement

- forward primer: GCCATCGCATCTGAAGTAAGC (SEQ ID NO: 5)

- reverse primer: GGTTGCTTTCTGTGTTAATCTGTGT (SEQ
ID NO: 6)

- reporter 1: TTCAGCGTGCTCACG (SEQ ID NO: 7)

- reporter 2: CTTCAGCATGCTCACG (SEQ ID NO: 8)

The PCR reaction solution was prepared with 12.5 µl of TaqMan™ Genotyping Master Mix (Cat No. 4371353), 1.25 µl of Genotyping Assay Mix (forward primer, reverse primer, reporter 1, reporter 2), and 10.25 µl of DNase-free water. Then, 1 µl (10 to 50 ng) of genomic DNA was added to the solution to make a total of 25 µl, and held at 95° C. for 10 minutes. Then, denaturation was performed at 92° C. for 15 seconds, and 50 cycles of anneal/extend at 60° C. for 1 minute were performed, so that genotyping was performed by performing a real-time PCR reaction using ViiA 7 Real-Time PCR System (Applied Biosystems, USA).

Meanwhile, immunohistochemical analysis was performed as in Example 4.

That is, whole staining of sections (4 µm thick) was performed via the BenchMark XT Staining system and ultraVIEW Universal DAB Detection Kit (Ventana Medical Systems, Inc., Tucson, AZ, USA). MUC1 and MUC4 expressions were evaluated using scientific microscopy as a percent multiplication of intensity by area, where staining was observed in epithelial glands as follows (0 to 300): 0 if no staining; 1+ if faint/barely perceptible partial staining; 2+ for weak to moderate staining; 3+ for strong staining. In cancer tissue, areas that were strongly stained were scored. Each sample was scored in a blinded manner by a single pathologist.

The expression levels of MUC4 in the presence of the above mutations according to immunohistochemical analysis are shown in Table 8 below.

TABLE 8

Analysis result of subjects with MUC4 mutations

| | Antrum of controls (n=5) | Autrum of Cancer tissue P value gastric (n=9) | cancer from gastric patients cancer patients (n=9) | P value |
|---|---|---|---|---|
| MUC4 expression level | 54.80 ± 53.51 | 26.11 ± 51.95 | 76.11 ± 59.99 | 0.187 |

As shown in Table 8, it was confirmed that subjects with MUC4 mutations showed a tendency for MUC4 expression to decrease in the antrum region of the stomach in the case of gastric cancer patients and to increase in gastric cancer tissue.

Through this, it was found that gastric cancer can be predicted or diagnosed in the presence of a MUC4 mutation even using genotyping, which can perform genetic analysis more efficiently and simply in terms of cost and time.

Overall, according to one embodiment of the present invention, in the case of gastric cancer patients, unlike normal controls, there is mutation in one of the specific 13 regions of the MUC4 gene (rs774527434, rs534579185, rs77250903, rs868067409, rs531395109, rs754808151, rs1304612772, rs774907241, rs771925912, rs745342765, rs148735556, rs11717039 and rs547775645). In particular, MUC4 expression in the subject having the MUC4 germline missense mutation (rs547775645 missense mutation) is downregulated in noncancerous gastric mucosa, resulting in inducing gastric cancer due to the loss of function of MUC4, and the expression of MUC4 in cancer tissues is increased compared to normal tissues. Through this, it is found that gastric cancer can be predicted or diagnosed using the detecting agent of the MUC4 gene mutation.

1. A method for predicting or diagnosing gastric cancer in a subject,
wherein the method comprises detecting a mutation of a mucin 4 (MUC4) gene,
wherein the mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on the MUC4 gene.

2. The method of claim 1, wherein the subject has a family history of gastric cancer.

3. The method of claim 1, wherein the mutation suppresses expression of the MUC4 gene in noncancerous gastric mucosa.

4. The method of claim 1, wherein the mutation increases expression of the MUC4 gene in gastric cancer tissue compared to normal gastric tissue.

5. The method of claim 1, wherein the mutation at the rs547775645 locus is a missense mutation.

6. The method of claim 1, wherein the mutation is one or more mutations selected from the group consisting of NM_018406.7:c.5375C>T, NM_018406.7:c.5005T>C, NM_018406.7:c.7658G>A, NM_018406.7:c.11180G>C, NM_018406.7:c.15884G>A, NM_018406.7:c.10673G>A, NM_018406.7:c.6064G>A, NM_018406.7:c.7648G>T, NM_018406.7:c.6638C>T, NM_018406.7:c.6640G>T, and NM_018406.7:c.3053G>C.

7. The method of claim 1, wherein the detecting is performed by one or more detecting agent selected from the group consisting of antisense oligonucleotide, primer pair, probe, antibody, peptide and polynucleotide that specifically binds to the mutation.

8. The method of claim 1, wherein the gastric cancer is one or more selected from the group consisting of diffuse-type gastric cancer, intestinal-type gastric cancer, and mixed-type gastric cancer.

9. The method of claim 1, wherein the subject is a subject suffering from one or more cancers selected from the group consisting of stomach adenocarcinoma (STAD), colorectal cancer (CRC) and endometrial cancer (uterine corpus endometrial cancer, UCEC).

10. A kit for predicting or diagnosing gastric cancer, which comprises a composition comprising a detecting agent of a mutation of a mucin 4 (MUC4) gene,
wherein the mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on the MUC4 gene.

11. The kit of claim 10, wherein the kit further comprises an instruction, and the instruction describes that the gastric cancer is predicted or diagnosed when the mutation is present at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on the detected MUC4 gene.

12. The kit of claim 10, wherein the kit is applied to the subject suffering from one or more cancers selected from the group consisting of stomach adenocarcinoma (STAD), colorectal cancer (CRC) and endometrial cancer (uterine corpus endometrial cancer, UCEC).

13. A method for providing information for predicting or diagnosing gastric cancer comprising:
extracting genomic DNA from a sample of a subject; and
detecting a mutation at one or more loci selected from the group consisting of rs774527434, rs534579185, rs77250903, rs868067409, rs531395109, rs754808151, rs1304612772, rs774907241, rs771925912, rs745342765, rs148735556, rs11717039 and rs547775645 on a mucin 4 (MUC4) gene in the extracted genomic DNA.

14. The method of claim 13, wherein the subject has a family history of gastric cancer.

15. The method of claim 13, wherein the detecting the mutation comprises:
reacting a primer specific to a contiguous nucleotide sequence selected from nucleotide sequences of the loci region; and
amplifying the reactant.

16. The method of claim 13, wherein the mutation is one or more mutations selected from the group consisting of NM_018406.7:c.5375C>T, NM_018406.7:c.5005T>C, NM_018406.7:c.7658G>A, NM_018406.7:c.11180G>C, NM_018406.7:c.15884G>A, NM_018406.7:c.10673G>A, NM_018406.7:c.6064G>A, NM_018406.7:c.7648G>T, NM_018406.7:c.6638C>T, NM_018406.7:c.6640G>T, and NM_018406.7:¢.3053G>C.

17. The method of claim 13, wherein the method determines the gastric cancer when the mutation of the MUC4 gene is detected after the detecting the mutation of the MUC4 gene.
